(19) United States
(12) Patent Application Publication
(10) Pub. No.: US 2011/0053270 A1
(43) Pub. Date: Mar. 3, 2011

(54) Patterning Hydrogels

(76) Inventors: Theresa Chang, Painted Post, NY (US); Robert Randall Hancock, JR., Corning, NY (US); Trista Nicole Hesch, Corning, NY (US); Michael Lesley Sorensen, Waverly, NY (US); Ruchirej Yongsunthon, Painted Post, NY (US)

(21) Appl. No.: 12/862,967

(22) Filed: Aug. 25, 2010

Related U.S. Application Data
(60) Provisional application No. 61/237,098, filed on Aug. 26, 2009.

(51) Int. Cl. B05D 3/06; C12N 5/00

(52) U.S. Cl. 435/402; 427/2.11

(57) ABSTRACT

A method for forming a pattern-coated substrate includes disposing a composition comprising a polysaccharide-based polymer on a substrate to generate a coated substrate. The polysaccharide-based polymer composition is substantially free of cross-linking monomers. The method further includes exposing a portion of the coated substrate to a first dose of UV radiation to induce crosslinking of the polysaccharide-based polymer, wherein a portion of the substrate is shielded from the ionizing radiation. The UV exposed coated substrate may be washed or hydrated to remove uncross-linked polysaccharide-based polymer.

### CROSS-REFERENCE TO RELATED APPLICATION

This application claims the benefit of priority under 35 U.S.C. §119(e) of U.S. Provisional Application Ser. No. 61/237098 filed on Aug. 26, 2009.

### FIELD

The present disclosure relates to methods of patterning hydrogels, particularly polysaccharide-based hydrogels, and articles, such as cell culture articles, having patterned hydrogels.

### BACKGROUND

Patterned hydro gels have been used in a variety of applications, including sensors, cell culture and tissue engineering. Due to their swelling behavior, which can change reversibly in response to stimuli such as temperature, pH, salinity and glucose concentration, hydrogels can serve as sensors or as a component of a sensor. Hydrogels are of interest in cell culture and assay and tissue engineering because their modulus and hydrophillicity more closely resemble tissue than plastics.

There are a number of methods for producing patterned hydrogels. Most of these methods involve photocuring monomers, with an initiator, through a photomask and subsequent washing away of unreacted monomers and initiators, and possibly polymerization inhibitors or other components or reagents that may be used in producing the hydrogel polymers.

### BRIEF SUMMARY

The present disclosure describes, among other things, patterned hydrogels formed from crosslinkable polymers. In various embodiments, the polymers, such as polysaccharide-based polymers, are photo-crosslinked without the use of photoinitiators, curable monomers, and harmful or toxic solvents or materials. Accordingly, washing steps to remove such components or reagents may be reduced or entirely eliminated.

In various embodiments, the present disclosure provides a method for forming a pattern-coated substrate. The method includes disposing a composition comprising a polysaccharide-based polymer on a substrate to generate a coated substrate. The polysaccharide-based polymer composition is substantially free of cross-linking monomers. The method further includes exposing a portion of the coated substrate to a first dose of UV radiation to induce crosslinking of the polysaccharide-based polymer, wherein a portion of the substrate is shielded from the ionizing radiation. The UV exposed coated substrate may be washed or hydrated to remove uncross-linked polysaccharide-based polymer.

The method may further include exposing at least a portion of the cross-linked polysaccharide-based polymer and at least a portion of the initially shielded coated substrate to a second dose of UV radiation to produce a coated substrate having areas of higher cross-link density and areas of lower cross-link density. Alternatively, or in addition, the method may further include pre-exposing to UV radiation, at least a portion of the coated substrate subsequently exposed to the first dose of ionizing radiation and at least a portion of the coated substrate subsequently shielded from the first dose of ionizing radiation to produce a coated substrate having areas of higher cross-link density and areas of lower cross-link density.

The method may be used to produce a variety of articles having patterned hydrogel layers. In some embodiments, the method is used to produce cell culture articles having patterned hydrogel layers.

Advantages of one or more of the various embodiments presented herein over prior methods for patterning hydrogels will be readily apparent to those of skill in the art based on the following detailed description when read in conjunction with the accompanying drawings.

### BRIEF DESCRIPTION OF THE DRAWINGS

FIGS. 1-3 are schematic illustrations of method of producing patterned hydrogel layers.

FIG. 4 is a schematic cross-sectional view of a cell culture article having a well having a surface coated with a patterned hydrogel layer. In FIG. 4A, the well is uncoated, while in FIGS. 4B-C the well, or a portion thereof, are coated.

FIG. 5A-C are optical micrographs of examples of patterned hydrogel surfaces produced in accordance with the methods described herein. In FIGS. 5A and 5C, the crystal violet staining is used to show cross-linked areas

FIG. 5D is an optical micrograph of a photomask used to create the patterned surface depicted in FIG. 5C.

FIGS. 6A-B provide atomic force microscopic images of examples of patterned hydrogel surfaces produced in accordance with the methods described herein.

FIG. 7A is an optical micrograph of a patterned surface produced in accordance with the methods described herein, with crystal violet staining used to show areas of crosslinking.

FIG. 7B is a schematic drawing of the patterned screening device used to create the patterned surface depicted in FIG. 7A and illustrate how the pattern was produced.

FIG. 8A is an optical micrograph of the intersection containing all heights of the pattern depicted in FIG. 7A.

FIG. 8B is an image produced by atomic force microscopy of the intersection containing all heights of the pattern depicted in FIG. 7A.

FIG. 9 is an optical micrograph is an optical micrograph of a patterned surface produced in accordance with the methods described herein, with crystal violet staining used to show areas of crosslinking.

FIG. 10 provides atomic force microscopic images of patterned surfaces produced in accordance with the methods described herein. The top two images depict the hydrated (A) and dry (B) images of a hydroxyethylcellulose (HEC) layer treated for 3 minutes with UV, revealing a 10× difference in height (7.3 μm swollen vs. 700 nm dry). The bottom 3 images depict height profiles for HEC samples that have been UV treated for 10 minutes (C), 5 minutes (D) and 3 minutes (E).

FIG. 11 is a bar graph of relative cell numbers for 24 hour attachment as determined by lactate dehydrogenase (LDH) assay.

The drawings in are not necessarily to scale. Like numbers used in the figures refer to like components, steps and the like. However, it will be understood that the use of a number to refer to a component in a given figure is not intended to limit the component in another figure labeled with the same number. In addition, the use of different numbers to refer to components is not intended to indicate that the different numbered components cannot be the same or similar.

### DETAILED DESCRIPTION

In the following detailed description, reference is made to the accompanying drawings that form a part hereof, and in which are shown by way of illustration several specific embodiments of devices, systems and methods. It is to be understood that other embodiments are contemplated and may be made without departing from the scope or spirit of the present disclosure. The following detailed description, therefore, is not to be taken in a limiting sense.

All scientific and technical terms used herein have meanings commonly used in the art unless otherwise specified. The definitions provided herein are to facilitate understanding of certain terms used frequently herein and are not meant to limit the scope of the present disclosure.

As used in this specification and the appended claims, the singular forms “a”, “an”, and “the” encompass embodiments having plural referents, unless the content clearly dictates otherwise.

As used in this specification and the appended claims, the term “or” is generally employed in its sense including “and/or” unless the content clearly dictates otherwise.

As used herein, “have”, “having”, “include”, “including”, “comprise”, “comprising” or the like are used in their open ended sense, and generally mean “including, but not limited to”. It will be understood that “consisting essentially of”, “consisting of”, and the like are subsumed in “comprising” and the like. Accordingly, composition comprising a polysaccharide-based polymer may be a composition consisting of, or consisting essentially of a polysaccharide-based polymer.

As used herein, “hydrogel” means a polymer that can absorb water in an amount greater than or equal to 30% of its dry weight. In many embodiments, a hydrogel can absorb water in an amount greater than or equal to 100% of its dry weight. It will be understood that the amount of water that a hydrogel polymer can absorb may vary depending on the degree that the polymer is crosslinked, where greater crosslinking often leads to less water absorption or swelling.

As used herein a “patterned hydrogel” means a hydrogel layer having a surface with intended topographical features.

As used herein, “polysaccharide-based polymer” means a polymer having a backbone of linked monosaccharide units. For example, a poly-glucose-based polymer is a polymer having a backbone of linked glucose units. By way of further example, a cellulose-based polymer is a polymer having a backbone of β(1→4) linked glucose units. It will be understood that pendant moieties of a polysaccharide-based polymer may be substituted, as desired, relative to the native monosaccharide. For example, hydroxyethylcellulose (HEC), hydroxypropylcellulose (HPC), methylcellulose (MC), and hydroxyproplymethylcellulose (HPMC) are all considered cellulose-based polymers.

The present disclosure describes, inter alia, patterned hydrogels formed from crosslinkable polymers. Typically, patterned hydrogels are formed by photocuring monomers, with an initiator, through a photomask and subsequent washing away of unreacted monomers and initiators, and possibly polymerization inhibitors or other components or reagents used to produce the hydrogel polymers. However, as described herein, it has been found that polymers, such as water soluble polysaccharide-based polymers can be photo-crosslinked to form patterned hydrogel surfaces. The water soluble polysaccharide-based polymers can cross-link when exposed to UV radiation without the use of photoinitiators and without the addition of cross-linking monomers. Accordingly, in some embodiments, washings steps to remove components or reagents, such as uncured monomers and initiators, may be reduced or entirely eliminated.

Any suitable water soluble, cross-linkable polysaccharide-based polymer may be employed to produce a patterned hydrogel as described herein. In various embodiments, the polysaccharide-based polymer is a poly-glucose-based polymer, such a cellulose-based polymer, a dextran-based polymer, an amylose-base polymer, or a starched based polymer such as hydroxyethyl starch. In some embodiments, the polysaccharide-based polymer is a poly-xylose-based polymer, such as a xylan-based polymer.

In various embodiments, a poly-glucose-based polymer has a structure according to

Formula I:

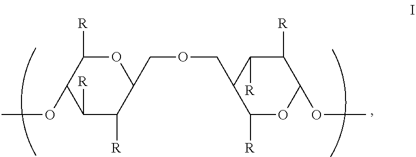

where each R is independently is:

(i) OX, where X is H, C₁-C₃ straight or branched alkyl, C₁-C₅ straight or branched alkoxy; or

(ii) YOX, where X is as described above and Y is C₁-C₃ alkyl.

For example, R may be OH, OCH₃, CH₂OCH₂CH₂OH, OCH₂CH₂OH, CH₂OCH(CH₃)OCH₂CH(CH₃)OH, OCH₂CH(CH₃)OH, CH₂OCH₃, or CH₂OCH₂CH(CH₃)OH.

In various embodiments, the above poly-glucose-based polymers may be hydrophobically modified, where a C₁₀ or greater alkyl, such as a C₁₆ or C₁₈ alkyl, is substituted via and ether linkage at one or more available hydroxyl groups.

It will be understood that other polysaccharide-based polymers may be similarly substituted. For example, poly-xylose-based monomers may be formed from xylose units as shown below in Formula II, where each R that is not part of the polymer backbone is independently as described above with regard to the poly-glucose-based polymer of Formula I.

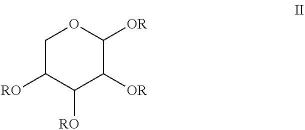

Polysaccharide-based polymers may be synthesized according to processes well known in the art or may be obtained from a suitable vendor, such as Sigma-Aldrich, Dow, or Aqualon.

In various embodiments the polysaccharide-based polymer is a neutral polymer. It is believed that charged polysaccharides may be too water soluble to form suitable hydrogels even after cross-linking; i.e., washing or hydration to remove uncrosslinked polymer may also result in removal of charged cross-linked polymer.

It will be understood that the cross-linking density may be affected by the molecular weight of the polymer employed. For example, it is believed that higher molecular weight polymers may achieve higher cross-linking densities than lower molecular weight polymers.

In some embodiments, the polysaccharide-based polymer is a cellulose-based polymer is selected from the group consisting of hydroxyethylcellulose (HEC), hydroxypropylcellulose (HPC), methylcellulose (MC), and hydroxyproplymethylcellulose (HPMC) or hydrophobically modified derivates thereof. Currently vendors including Sigma-Aldrich, Dow, and Aqualon offer a variety of cellulose polymers. By way of example, and with reference to HEC, (i) SIGMA-Aldrich offers HEC having an average molecular weight of about 90 kDa, about 250 kDa, about 720 kDa, or about 1,300 kDa, (ii) Dow offers a variety of HEC products having 1% Brookfield Viscosities ranging from about 1100 to about 6000 cP and offers an HMHEC that is a hydrophobe modified HEC (CELLOSIZE HMHEC 500) having an HEC backbone with pendant hydrophobic groups, and (iii) Aqualon offers HEC as Natrosol 250 and HMHEC as PolySurf 67, having pendant cetyl groups.

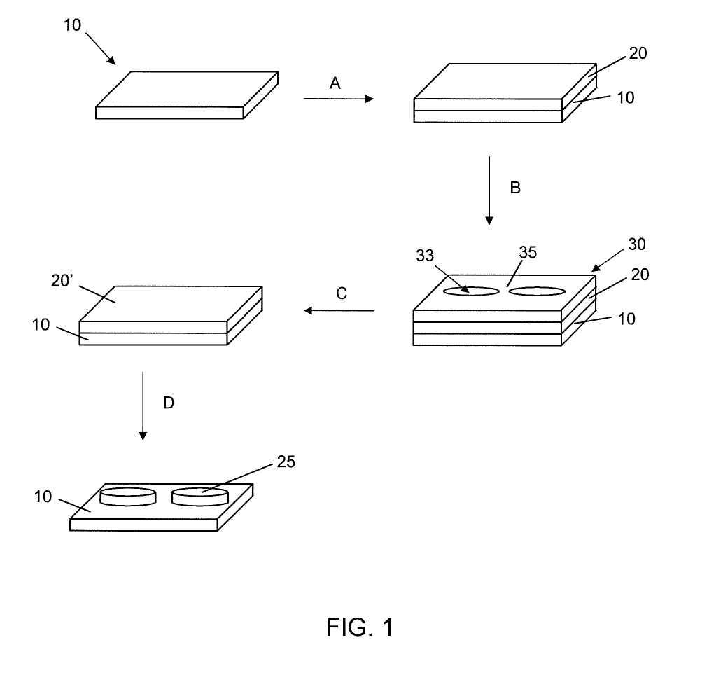

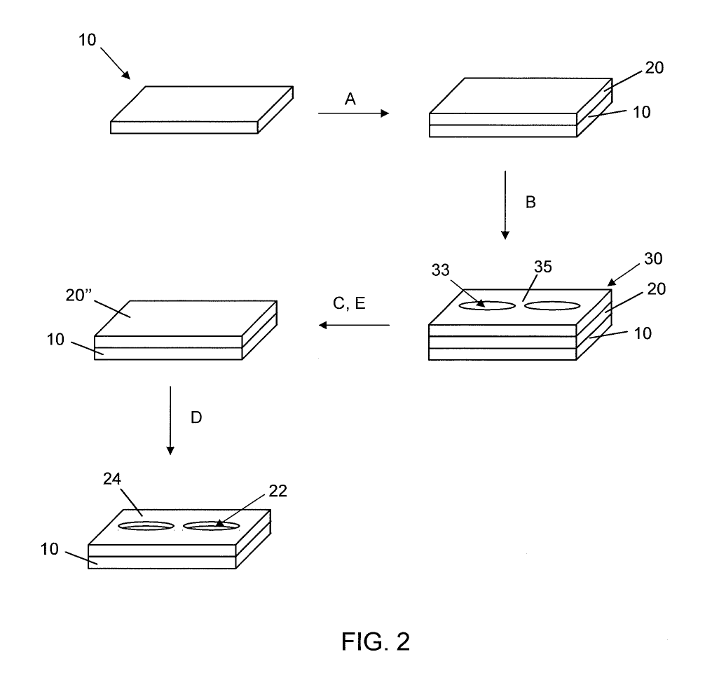

Referring now to FIGS. 1-3, various methods for forming patterned hydrogels are shown in schematic form. As shown in FIG. 1 and FIG. 2, at least a portion of a surface of a substrate 10 is coated with a water soluble polysaccharide-based polymer 20 (Step A). Then, a patterned UV shield 30 having regions that allow transmission of UV light 33 and regions that bock transmission of UV light 35 is disposed over the polysaccharide-based polymer layer 20, and the resulting assembly is exposed to UV radiation (Step B). The portion of the polysaccharide-based polymer layer 20 that is beneath the regions of the patterned shield that allows transmission of UV light 33 is exposed to UV radiation, and the portion of the polysaccharide-based polymer layer 20 that is beneath the regions of the patterned shield that blocks of UV light 35 is shielded from UV radiation. Those portions of the layer 20 exposed to UV radiation undergo cross-linking, while the shielded portions do not undergo UV-induced crosslinking. The patterned shield 30 is removed (Step C), and at this point the schematic methods depicted in FIGS. 1-2 differ.

In the method depicted in FIG. 1, the patterned shield 30 is removed (Step C) and the resultant polysaccharide-based polymer layer 20′ having cross-linked regions and non-cross linked regions is hydrated or washed to remove the uncrosslinked polymer leaving the patterned cross-linked polymer with patterned features 25 (Step D).

In the method depicted in FIG. 2, the patterned shield 30 is removed (Step C) and the entire resultant polysaccharide-based polymer layer is exposed to further UV radiation (Step E). The resulting polymeric layer 20″ has regions with a higher amount of crosslinking (those areas exposed with shield in place and with shield removed) and regions with a lower amount of crosslinking (those areas exposed only with shield removed). Upon hydration (Step D) the areas with higher cross-link density 22 do not swell as much as those areas with lower cross-link density 24, leaving a patterned layer.

It will be understood that the method depicted in FIG. 2 could be performed in a different order. For example, the entire polysaccharide-based polymer layer could be exposed to UV light prior to disposing the patterned shield over the layer. In addition to, or alternatively to, exposing the entire layer to UV light, it will be understood that more than more than one shield may be used, where the shields have overlapping areas that allow UV transmission to give differential cross-linking densities.

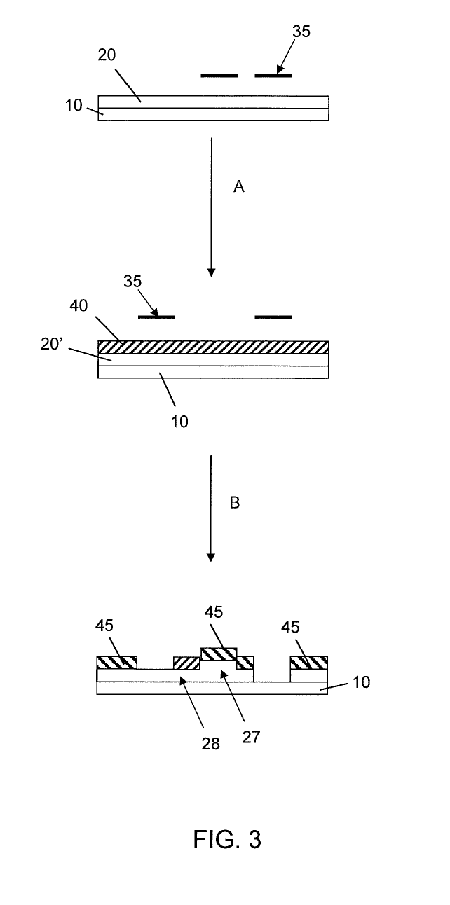

Referring now to FIG. 3, an overview of a method employing two layers including polysaccharide-based polymers is shown. In the depicted embodiment, a polysaccharide-based polymer hydrogel layer 20 disposed on a substrate 10 is exposed to UV radiation, with a portion of the layer 20 being shielded from the UV radiation by non-UV transmitting portions 35 of a patterned UV shield. A second polysaccharide-based polymer hydrogel layer 40 is disposed on the UV irradiated first layer 20′, and the second layer 40 is exposed to UV radiation, with a portion of the layer 40 being shielded from the UV radiation by non-UV transmitting portions 35 of a patterned UV shield (Step A). The second layer 40 may be of the same composition or of a different composition than the first layer 20. Upon hydration or washing (Step B), the resulting topographical surface may include areas 28 where the first layer is exposed (where second layer was shielded from UV), areas 18 where the substrate 10 is exposed (where first and second layers were shielded from UV), and areas 45 where the second layer is exposed (where second layer was exposed to UV). The first hydrogel layer may have areas of higher 28 and lower 27 crosslink densities, depending on whether the areas were shielded from or exposed to the first dose of UV radiation. The resultant surface may thus have a variety of heights and exposed materials when hydrated. It will be understood that a variety of other surface topographies and characteristics may be obtained by employing additional polysaccharide-based polymer layers, by exposing one or more layers to differing patterns shields and thus differential amounts of UV radiation, and the like. Further, the composition of a layer, the thickness of a layer, and the amount of UV light to which a layer, or portion thereof, is exposed can affect the surface topography or properties.

A polysaccharide-based polymer layer may be disposed on a surface of a substrate or underlying polysaccharide-based polymer layer in any suitable manner. For example, the polysaccharide-based polymer may be placed on the substrate as a dry powder, may be poured or cast onto the substrate as a solution, gel or suspension, or the like. As the polysaccharide-based polymers that form hydrogels tend to be water soluble, water may be used as the solvent for creating a solution, gel or suspension for disposing on the substrate surface. Of course other solvents may also be employed.

The polysaccharide-based polymer composition may be suspended or dissolved in the solvent at any suitable concentration. Preferably, the composition uniformly coats or covers the substrate or underlying layer, or desired portion thereof It will be understood that the concentration and thickness of the polysaccharide-based polymer layer can be varied to achieve the desired properties of the resulting patterned hydrogel layer. In various embodiments, the polysaccharide-based polymer is suspended or dissolved in a solvent at a concentration of between 0.005% and 20% by weight. For example, the concentration of polysaccharide-based polymer may be between 0.01% and 10% by weight, between 0.1% and 1%, between 0.1% and 0.5%, about 0.2%, or the like.

The polysaccharide-based polymer compositions are substantially free of cross-linking monomers. By way of example, a polysaccharide-based polymer composition having less than about 1% by weight, less than 0.5% by weight, less than 0.1% by weight, less than 0.05% by weight, or less than 0.01% by weight crosslinking monomer would be considered to be substantially free of crosslinking monomer. Similarly, the polysaccharide-based polymer compositions may be substantially free of photoinitiators.

In various embodiments, the solution, suspension or gel is evaporated to dryness prior to UV treatment. In some embodiments, the solution, suspension, gel, or the like is dried such that it contains less than 5%, less than 2%, or less than 1% water by weight. Heat or vacuum may be used to facilitate evaporation. For example, the coated substrate may be placed at between about 40° C. and about 70° C., or at about 60° C. A solvent more volatile than water may be used to speed the evaporation process.

Any suitable patterned UV screen may be used to subject portions of the polysaccharide-based polymer layer to UV radiation. The patterned UV screen may be a photomask or other opaque plate with holes or transparencies to allow UV light to transmit through in a defined pattern. By way of example, a photomask may be formed from steel with UV transparent openings or patterned voids or may be formed from quartz with UV non-transparent Ni or Fe patterns.

The patterned UV screen can be placed in direct contact with the cross-linkable polysaccharide layer or in close proximity to the layer. Typically, the closer the UV screen is placed to the cross-linkable polysaccharide-based polymer, the better the resolution of the pattern achieved.

The cross-linkable polysaccharide-based polymer layer, with or without the patterned UV screen in place, can be exposed to any suitable amount of UV light. The intensity of the UV light or time of exposure to UV can be varied to achieve a desired amount of cross-linking. In many embodiments, a cross-linkable polysaccharide-based polymer layer is exposed to between about 50 and about 300 mJ/cm² UV radiation for a time of between about 1 minute and about 20 minutes.

The cross-linkable polysaccharide-based polymer layer may be disposed on any suitable substrate. It will be understood that the substrate may vary depending on the ultimate utility of the apparatus on which the patterned hydrogel is coated. Examples of suitable substrates include ceramic substrates, glass substrates, plastic or other polymeric substrates, or combinations thereof In some embodiments, the substrate is a glass materials such as soda-lime glass, pyrex glass, vycor glass, quartz glass; silicon. In some embodiments, the substrate is a plastic or polymers including dendritic polymers, such as poly(vinyl chloride), poly(vinyl alcohol), poly(methyl methacrylate), poly(vinyl acetate-co-maleic anhydride), poly(dimethylsiloxane) monomethacrylate, cyclic olefin polymers, fluorocarbon polymers, polystyrenes, polypropylene, polyethyleneimine; copolymers such as poly(vinyl acetate-co-maleic anhydride), poly(styrene-co-maleic anhydride), poly(ethylene-co-acrylic acid) or derivatives of these or the like.

The substrate may be treated or coated to enhance the interaction between the substrate surface and the coated polysaccharide-based polymer layer or to impart a desirable characteristic to the surface. For example, the surface of the substrate may be activated via ionization, heating, photochemical activation, oxidizing acids, sintering, physical vapor deposition, chemical vapor deposition, and etching with strong organic solvents. In some embodiments, the substrate surface is plasma or corona treated.

Any article (i.e., device or apparatus) employing patterned hydrogels can be coated in accordance with the teachings presented herein. In various embodiments, a cell culture article includes a patterned polysaccharide-based hydrogel layer as described herein. Examples of cell culture articles to which a patterned hydrogel layer may be applied include single and multi-well plates, such as 6, 12, 96, 384, and 1536 well plates, jars, petri dishes, flasks, multi-layered flasks, beakers, plates, roller bottles, slides, such as chambered and multichambered culture slides, tubes, cover slips, bags, membranes, hollow fibers, beads and microcarriers, cups, spinner bottles, perfusion chambers, bioreactors, CellSTACK® and fermenters.

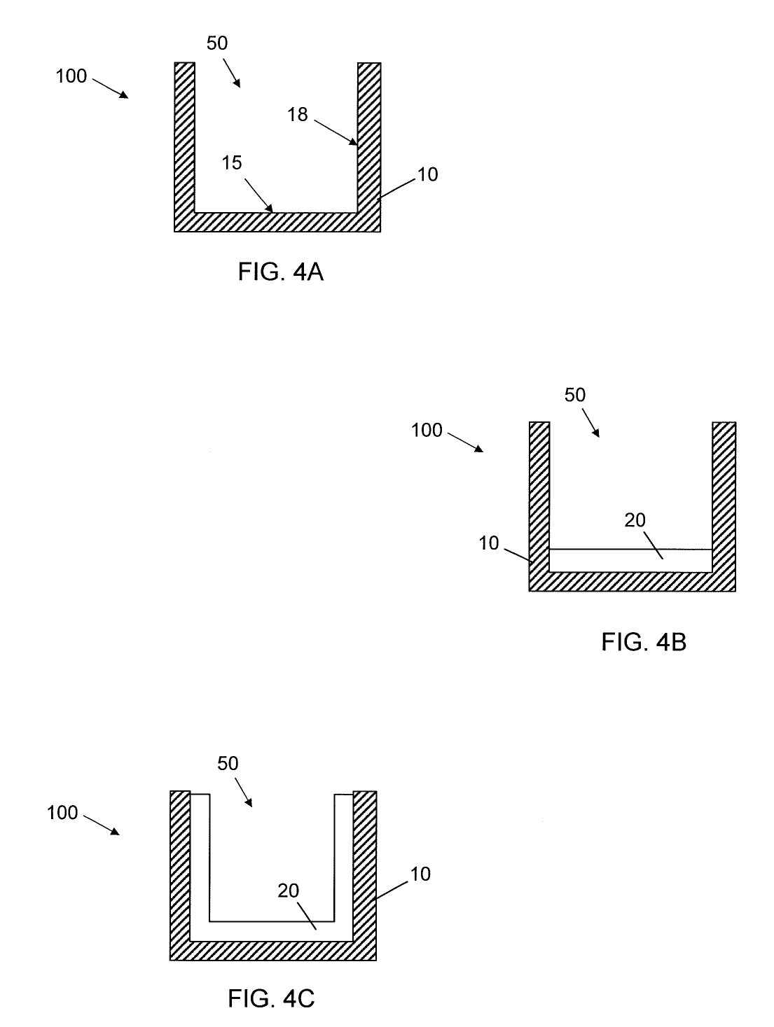

In numerous embodiments, the cell culture article surface to which a patterned hydrogel layer is applied is a surface within a well. Examples of cell culture articles having a well include plates, flasks, beakers, bottles, bags, chambers, fermentors, and the like. Referring now to FIG. 4, a cell culture article 100 formed from a substrate or base material 10 may include one or more wells 50. Well 50 includes a sidewall 18 and a surface 15. Referring to FIG. 4B-C, a patterned hydrogel coating 20 may be disposed on surface 15 or sidewalls 18, or a portion thereof

A cell culture article having a patterned hydrogel layer as described above may be seeded with cells. The cells may be of any cell type. For example, the cells may be connective tissue cells, epithelial cells, endothelial cells, hepatocytes, skeletal or smooth muscle cells, heart muscle cells, intestinal cells, kidney cells, or cells from other organs, stem cells, islet cells, blood vessel cells, lymphocytes, cancer cells, primary cells, cell lines, or the like. The cells may be mammalian cells, preferably human cells, but may also be non-mammalian cells such as bacterial, yeast, or plant cells. A hydrogel patterned surface may be particularly suitable for culturing adherent cells.

In an aspect (1) a method for forming a pattern-coated substrate, comprising: disposing a composition comprising a polysaccharide-based polymer on a substrate to generate a coated substrate, wherein the composition is substantially free of cross-linking monomers; exposing a portion of the coated substrate to a first dose of ultraviolet radiation to induce crosslinking of the polysaccharide-based polymer, wherein a portion of the substrate is shielded from the ionizing radiation is provided. In an aspect (2) the method of aspect 1 is provided, further comprising washing the coated substrate to remove uncross-linked polysaccharide-based polymer. In an aspect (3) the method of aspect 1 or 2 is provided, further comprising exposing at least a portion of the cross-linked polysaccharide-based polymer and at least a portion of the initially shielded coated substrate to a second dose of ultraviolet radiation to produce a coated substrate having areas of higher cross-link density and areas of lower cross-link density. In an aspect (4) the method of any one of aspects 1-3 is provided, further comprising pre-exposing to ionizing radiation, at least a portion of the coated substrate subsequently exposed to the first dose of ultraviolet radiation and at least a portion of the coated substrate subsequently shielded from the first dose of ultraviolet radiation to produce a coated substrate having areas of higher cross-link density and areas of lower cross-link density. In an aspect (5) the method of any one of aspects 1-4 is provide, wherein the polysaccharide-based polymer is a poly-glucose-based polymer or a poly-xylose-based polymer. In an aspect (6) the method of any one of aspects 1-5 is provided, wherein the poly-glucose-based polymer is selected from a cellulose-based polymer, a dextran-based polymer, or an amylase-based polymer. In an aspect (7), the method of any one of aspects 1-6 is provided, wherein the polysaccharide-based polymer is selected from the group consisting of hydroxypropylcellulose, methylcellulose, and hydroxyproplymethylcellulose, hydroxyethylcellulose, amylose, dextran, and xylan, or hydrophobically modified derivates thereof In an aspect (8), the method of any one of aspects 1-7 is provided wherein a cell culture article provides the substrate.

In a further aspect (9), a method for pattern-coating a surface of a cell culture article, comprising: disposing a composition comprising a polysaccharide-based polymer on a surface of the article to generate a coated surface, wherein the composition is substantially free of cross-linking monomers; exposing a portion of the coated surface to ultraviolet radiation to induce crosslinking of the polysaccharide-based polymer, wherein a portion of the substrate is shielded from the ionizing radiation is provided. In an aspect (10), the method of aspect 9 is provided, further comprising washing the coated substrate to remove uncross-linked polysaccharide-based polymer. In an aspect (11), the method of aspect 9 or 10 is provided, further comprising exposing at least a portion of the cross-linked polysaccharide-based polymer and at least a portion of the initially shielded coated substrate to a second dose of ultraviolet radiation to produce a coated substrate having areas of higher cross-link density and areas of lower cross-link density. In an aspect (12), the method of any one of aspects 9-11 is provided further comprising pre-exposing to ionizing radiation, at least a portion of the coated substrate subsequently exposed to the first dose of ultraviolet radiation and at least a portion of the coated substrate subsequently shielded from the first dose of ultraviolet radiation to produce a coated substrate having areas of higher cross-link density and areas of lower cross-link density. In an aspect (13), the method of any one of aspects 9-12 is provided, wherein the polysaccharide-based polymer is a poly-glucose-based polymer or a poly-xylose-based polymer. In an aspect (14) the method of any one of aspects 9-13 is provided, wherein the poly-glucose-based polymer is selected from a cellulose-based polymer, a dextran-based polymer, or an amylase-based polymer. In an aspect (15) the method of any one of aspects 9-14 is provided, wherein the polysaccharide-based polymer is selected from the group consisting of hydroxypropylcellulose, methylcellulose, and hydroxyproplymethylcellulose, hydroxyethylcellulose, amylose, dextran, and xylan, or hydrophobically modified derivates thereof In a further aspect (16), a cell culture article produced by the method of any one of aspects 9-15 is provided.

In a further aspect (17) a cell culture article comprising: a patterned surface for culturing cells, the surface formed from a coating consisting essentially of a cross-linked polysaccharide-based polymer, wherein the coating is free of cross linking monomers is provided. In an aspect (18), the article of aspect 17, wherein the polysaccharide-based polymer is a poly-glucose-based polymer or a poly-xylose-based polymer is provided. In an aspect (19) article of claim 17 or 18 is provided, wherein the poly-glucose-based polymer is selected from a cellulose-based polymer, a dextran-based polymer, or an amylase-based polymer. In an aspect, the article of any one of aspects 17-19 is provided, wherein the polysaccharide-based polymer is selected from the group consisting of hydroxypropylcellulose, methylcellulose, and hydroxyproplymethylcellulose, hydroxyethylcellulose, amylose, dextran, and xylan, or hydrophobically modified derivates thereof

In the following, non-limiting examples are presented, which describe various embodiments of representative methods for producing patterned hydrogel surfaces and articles produced by the methods.

### EXAMPLES

### Example 1

### Hydrogel on Substrate

A solution of PolySurf67 (Aqualon), a hydrophobically modified hydroxyethylcellulose (HMHEC) having cetyl substitution, or another water-soluble, crosslinkable polymer (hydroxyethylcellulose (HEC), dextran, xylan, or 2-hydroxyethyl cellulose), in water (0.2 wt %, 1-2 mL) was cast into a 6 well polystyrene plate. According to Aqualon's product brochure (Polymers for Hair and Skin Care—Aqueous system solutions for cosmetic and personal care products, 250-50 F, REV. 02-08), HMHEC is made in a two-step reaction, with the first being a standard reaction of alkali-cellulose with ethylene oxide to produce HEC, and the second being a cetyl substitution, which provides the hydrophobic end groups. PolySurf67 has a Brookfield viscosity (1% solution, cps) of 8000-14,000 and has an average molecular weight of 550,000.

The solution of PolySurf67 or other water-soluble, crosslinkable polymer was allowed to evaporate to dryness (60° C.) and UV treated with a UV ozone bulb in ambient conditions (185/254 nm) approximately 1.5 inches from the coating for 10 minutes. Upon removal of the photomask [PPM Photomask Inc. (4950 Fisher Street, Montreal, QC., Canada H4T 1J6)], there was no apparent change in the film after UV irradiation. The features only appeared upon addition of water or if the films were exposed to areas of high humidity. When water was added, the crosslinked regions swelled, and the uncrosslinked portions dissolved away. The dissolution process could be increased by elevating the temperature (40-60° C.). At 60° C., only the crosslinked hydrogel remained on the surface in the presence of water.

In another example, 1 mL TMOS was added to 100 mL of a 0.5% HEC solution (2-hydroxyethyl cellulose, Sigma Aldrich, MW=250 kDa) while stirring. This was designated 2× TMOS/HEC. However, any suitable amount of TMOS can be added to an HEC solution. For example, 0.05× TMOS to 4× TMOS HEC solutions can readily be prepared, if necessary. The 2× TMOS/HEC solution was coated on top of the dried HEC layer and exposed to 10 minutes of UV light. The source is about 4 inches away from the 6 well plate bottom surface. The HEC can be exposed to UV light various times to get various patterns on it. Patterned masks were used to block UV light coming through during UV treatment to provide the various patterns.

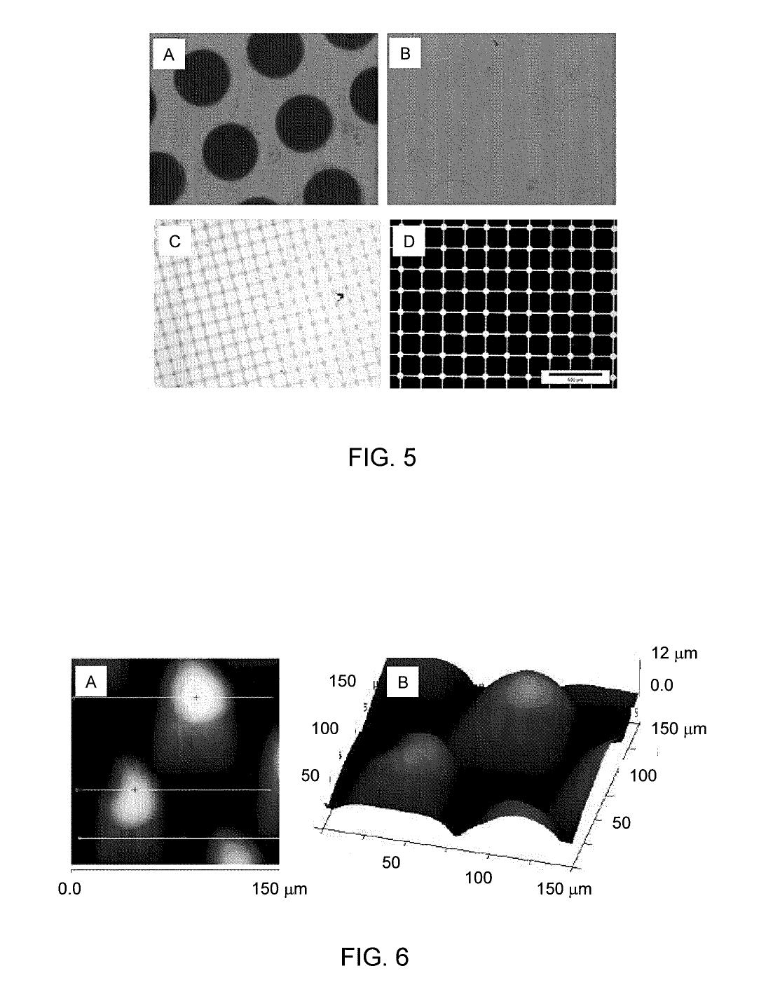

In the images shown in FIGS. 5A and 5B, a photomask with round openings was used, and the HEC exposed to UV radiation exhibited cross-linking, as evidenced by the crystal violet staining in FIG. 1A. As shown in the image in FIG. 5B, in which no crystal violet stain was employed, the cross-linked area has a topographical height, as evidenced by the shadows.

The images in FIGS. 5C and 5D are of a patterned coated hydrogel (5C), with crystal violet, and the photomask used to generate the pattern (5D). The results presented in FIG. 5C again confirms that polysaccharide-based polymers are capable of cross-linking when exposed to UV radiation. Similar results were also observed with other water-soluble, crosslinkable polymers, including hydroxyethylcellulose (HEC), dextran, xylan, and 2-hydroxyethyl cellulose (data not shown).

It was observed that the lateral feature sizes available are limited based on a variety of parameters including the quality of the cast film, the distance between the film and mask, and how well scattering can be prevented. For example, using a UV mask with line widths of 20 μm resulted in features with ˜50 μm (as visualized by crystal violet staining) It is believed that the resolution can be improved by filtering the polymer solution before casting to get rid of any dust or particulates that may scatter the light. Furthermore, the unevenness of the film from the presence of particulates may also increase the gap between the mask and the film, resulting in incomplete blocking of the UV light particularly at the boundaries thereby decreasing the resolution of the resulting pattern. It may also be possible to improve the resolution of the patterns by the use of a collimator. The experiments that resulted in the images presented in FIGS. 5A-D were conducted without the use of a collimator.

The UV crosslinked regions can be stained with crystal violet for easier visualization by optical microscopy (FIG. 5) or if the feature sizes are too small the topography can be seen with Atomic Force Microscopy (FIG. 6).

The images presented in FIGS. 6A-B show examples of patterned hydrogel surfaces imaged by atomic force microscopy (AFM) showing that the small feature sizes can be obtained by UV radiation of hydrogel polymers. The feature sizes shown in FIGS. 6A-B are in the range of about 50 μm to 75 μm.

### Example 2

### Hydrogel Topography

A cast film as described in EXAMPLE 1 was UV treated in the presence of a photomask (patterned quartz from PPM Photomask). The photomask was then removed and the film was subjected to another dose of UV irradiation (for a further 1-4 minutes). As with the HEC films in EXAMPLE 1, the features appeared upon hydration, where the more crosslinked areas (those subjected to UV before and after removal of the mask) appeared as depressions because they do not swell as much as the more lightly crosslinked areas (those areas only subjected to UV after removal of the mask). One will appreciate that the relative height of the different areas can be balanced by changing either the amount of material coated or the UV exposure time (see, e.g., EXAMPLE 4 below).

### Example 3

### Multilayered Structures

It is also possible to use the procedures described in EXAMPLE 1 or EXAMPLE 2 to form multilayered or multifunctional structures. For example, a first polymer film may be cast and treated with UV and a photomask. However, instead of washing or hydrating the film, a second layer of polymer can be cast on top of the first layer. The second layer can be treated using another photomask and upon removal of the mask, hydration and washing, the surface can include: 1) bare substrate if neither the first or second layer was exposed to UV, 2) the second polymer layer where the second layer was exposed to UV treatment (although there could be height differences depending on whether the first layer was UV treated), and 3) the first polymer layer exposed in areas where the first polymer was subjected to UV treatment but the second layer wasn't and was therefore washed away.

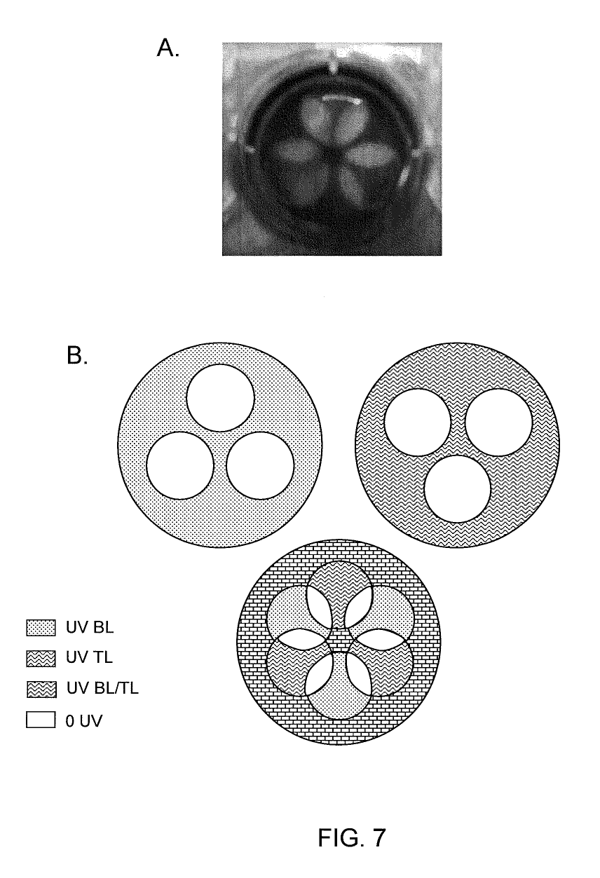

An example of a patterned hydrogel produced according to such a method is shown in FIG. 7. In this case, glass cover slips were used as photomasks and were placed on top of HEC polymer films. The cover slips were placed (12:00=0°) at 0°, 120° and 240°, and the film was treated with UV (180 mJ/cm², 1.5 inches from screen, 10 min) A second HEC layer was deposited on top of the first layer, and the cover slips were staggered from the first set, placed at 30°, 180°, 300°. The substrate was subjected to UV treatment accordingly (180 mJ/cm², 1.5 inches from screen, 10 min), hydrated by submersion in 2-5 ml of water, and stained with crystal violet. The sample was then dried to dryness (excess water was removed by shaking, and the sample was then placed in 60-70° C. oven until dry) and the topography measured with AFM using a Bioscope II (Veeco). AFM analysis is possible to perform as the shrinking of the coating upon drying is not 100% reversible and it does not return to a completely smooth film. The resulting AFM image is shown in FIG. 7A. Schematic diagrams indicating the patterning of UV radiation of the bottom layer, top layer, and overall coating are shown in FIG. 7B; where UV BL refers to the UV treated portion of the bottom layer, UV TL refers to the UV treated portion of the top layer, UV BL/TL refers to portions of both the top and bottom layers that were subjected to UV radiation, and 0 UV refers to areas of the top and bottom layers that were shielded from UV radiation.

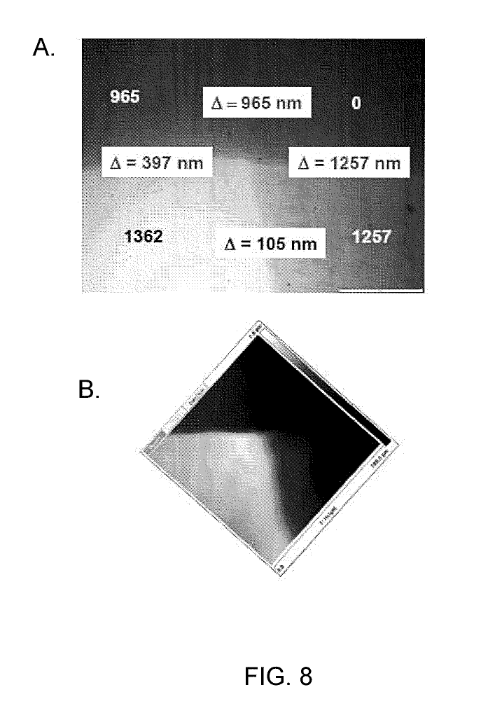

With the two coating layers used, areas with four distinct heights were observed (see FIG. 8). As shown in FIG. 8, it is possible to image and measure the relative dry height difference between the 4 sections (FIG. 8) by looking at the height differences at the boundaries of the sections. FIG. 8A is an optical micrograph of an intersection containing all heights of the pattern depicted in FIG. 7A and FIG. 7B. FIG. 8B is an AFM image of the same region. Since crystal violet adsorbs more to regions with higher UV exposure, the optical graph is indicative of the relative heights of the 4 regions. This was confirmed by AFM as the height differences (marked as Δ at the boundaries). The height differences measured are in blue whereas the Δ value in black (105 nm) is calculated as the boundary is less resolved than the other boundaries.

One will appreciate that the polymers employed in the first and second layers can be the same or different. For example, differences in hydrophobicity can be achieved by using HEC for one layer and hydrophobically modified HEC for the other.

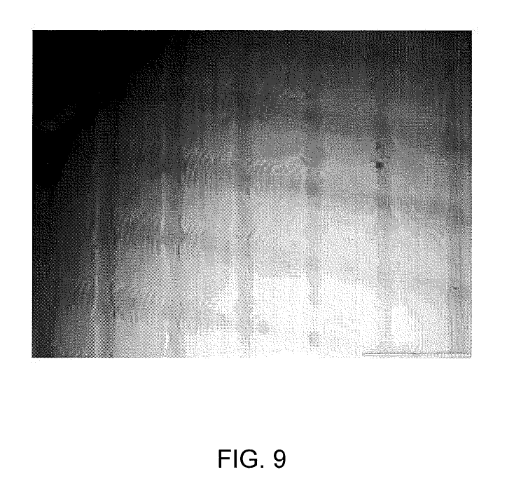

As shown in FIG. 9, this technique can work with smaller feature sizes. To produce the two-layered pattern shown in the optical micrograph in FIG. 9, a photomask having a pattern consisting of lines with circles along the lines at regular intervals was used. A first HEC hydrogel layer was coated on a polystyrene substrate and subjected to UV light (180 mJ/cm², 1.5 inches from light source, 10 min) with the photomask in place. A second HEC hydrogel layer was then coated on the first layer with the photomask placed approximately orthogonally to the first placement and the second layer was subjected to UV light, as above, with the photomask in place. The layer depicted in FIG. 9 resulted. As shown, feature sizes as small as 20 μm were obtainable. Of course, smaller feature sizes should be readily obtainable by those of skill in the art using the teaching presented herein.

### Example 4

### UV Treatment Time and Film Thickness

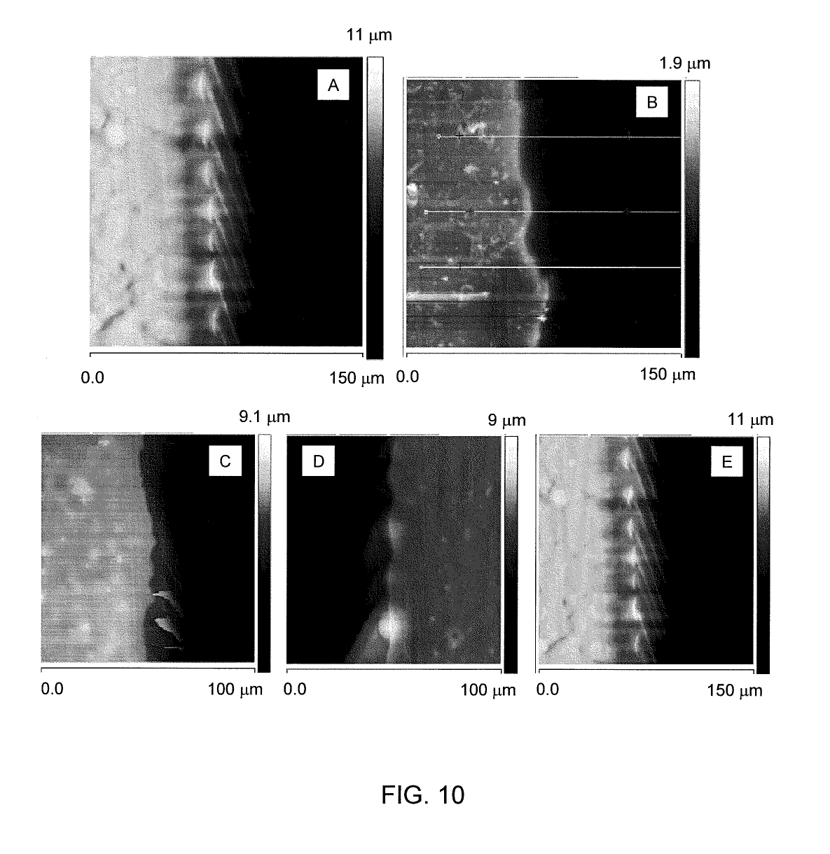

The film thickness can be varied by altering the amount of polymer deposited (varying concentration of casting solution) onto the substrate as well as changing the UV exposure time. For example, samples with the same amount of material (0.2 wt %, 1 mL, ˜9.5 cm²) were subjected to 2, 3, 5 and 10 minutes of UV treatment and the hydrated height measured by atomic force microscopy (FIG. 10). The dry and hydrated height was measured for the 3 minute sample and it was found that the hydrogel swelled to 10× its height upon hydration (from 700 nm to 7.3 μm). We have found that the 10 minute and 5 minute samples were virtually identical in their swelling behavior (˜3.5 μm). The height of the 2 minute sample was beyond the measurable instrument range, with a height >11 μm.

In FIG. 10, the top two images depict the hydrated (A) and dry (B) images of a hydroxyethylcellulose (HEC) layer treated for 3 minutes with UV, revealing a 10× difference in height (7.3 μm swollen vs. 700 nm dry). The bottom 3 images depict height profiles for HEC samples that have been UV treated for 10 minutes (C), 5 minutes (D) and 3 minutes (E).

### Example 5

### Cell Culture and Assay

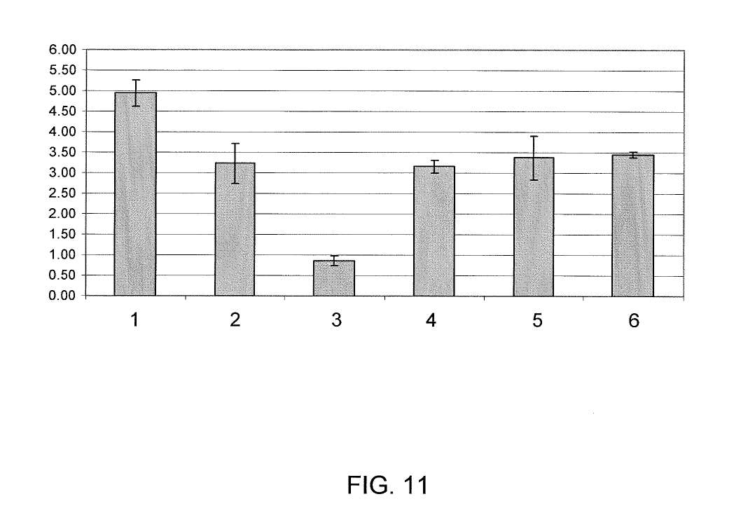

Briefly, HepG2/C3A cells (ATCC # CRL-10741) were cultured in Eagle's Minimum Essential Medium (ATCC # 30-2003) supplemented with 10% Fetal Bovine Serum (Invitrogen # 16000-077) and 1% Penicillin-Streptomycin (Invitrogen # 15140-155). Cells were incubated at 37° C., in 5% CO₂ and 95% relative humidity. Cells were seeded on the modified cellulose coatings (n=3) at 5000 cells in 100 μL media per well in 96 well microplate format. Media was replaced daily. A Lactate Dehydrogenase (LDH) assay (Promega CytoTox 96® Non-Radioactive Cytotoxicity Assay # G1780) was performed to access cell attachment. The cells were lysed according to manufacturer's instructions and the amount of LDH released, which is theoretically directly proportional to the number of cells present, was measured. Results are reported in FIG. 11.

### Example 6

### Patterning For Enhancement of Cell Attachment

We have also demonstrated that patterning can be used to improve cell adhesion. HepG2/C3A cells were seeded on HEC/TMOS coatings (tetramethylorthosilicate added to aid cell attachment) using collagen I and Matrigel as controls in the presence of serum. The relative cell number was measured after 24 hours using a LDH (lactate dehydrogenase) assay (FIG. 11). The Y-axis in FIG. 11 depicts the average optical density at 490 nanometers. On the X-axis, 1 refers to a collagen-1 coated surface, 2 refers to MARTIGEL coated surface, 3 refers to un-patterned HEC/TMOS surface, and 4-6 refer to patterned HEC/TMOS surfaces where each or 4, 5, and 6 are uniquely patterned. FIG. 11 shows cell attachment to the patterned surfaces (4-6) with patterns in the sub micron range. Cell attachment achieved with patterned surfaces (4-6) was comparable to cell attachment shown on Matrigel™. However, the non-patterned (UV-treated) substrate retained only ⅓ as many cells. While HEC/TMOS coatings were employed in this Example, HEC/TEOS coatings would also be expected to work well for cell culture in light of the findings presented herein.

Thus, embodiments of PATTERNING HYDROGELS are disclosed. One skilled in the art will appreciate that the arrays, compositions, kits and methods described herein can be practiced with embodiments other than those disclosed. The disclosed embodiments are presented for purposes of illustration and not limitation.

What is claimed is:

1. A method for forming a pattern-coated substrate, comprising:
disposing a composition comprising a polysaccharide-based polymer on a substrate to generate a coated substrate, wherein the composition is substantially free of cross-linking monomers;
exposing a portion of the coated substrate to a first dose of ultraviolet radiation to induce crosslinking of the polysaccharide-based polymer, wherein a portion of the substrate is shielded from the ionizing radiation.

2. The method of claim 1, further comprising washing the coated substrate to remove uncross-linked polysaccharide-based polymer.

3. The method of claim 1, further comprising exposing at least a portion of the cross-linked polysaccharide-based polymer and at least a portion of the initially shielded coated substrate to a second dose of ultraviolet radiation to produce a coated substrate having areas of higher cross-link density and areas of lower cross-link density.

4. The method of claim 1, further comprising pre-exposing to ionizing radiation, at least a portion of the coated substrate subsequently exposed to the first dose of ultraviolet radiation and at least a portion of the coated substrate subsequently shielded from the first dose of ultraviolet radiation to produce a coated substrate having areas of higher cross-link density and areas of lower cross-link density.

5. The method of claim 1, wherein the polysaccharide-based polymer is a poly-glucose-based polymer or a poly-xylose-based polymer.

6. The method of claim 5, wherein the poly-glucose-based polymer is selected from a cellulose-based polymer, a dextran-based polymer, or an amylase-based polymer.

7. The method of claim 1, wherein the polysaccharide-based polymer is selected from the group consisting of hydroxypropylcellulose, methylcellulose, and hydroxyproplymethylcellulose, hydroxyethylcellulose, amylose, dextran, and xylan, or hydrophobically modified derivates thereof.

8. The method of claim 1, wherein a cell culture article provides the substrate.

9. A method for pattern-coating a surface of a cell culture article, comprising:
disposing a composition comprising a polysaccharide-based polymer on a surface of the article to generate a coated surface, wherein the composition is substantially free of cross-linking monomers;
exposing a portion of the coated surface to ultraviolet radiation to induce crosslinking of the polysaccharide-based polymer, wherein a portion of the substrate is shielded from the ionizing radiation.

10. The method of claim 9, further comprising washing the coated substrate to remove uncross-linked polysaccharide-based polymer.

11. The method of claim 9, further comprising exposing at least a portion of the cross-linked polysaccharide-based polymer and at least a portion of the initially shielded coated substrate to a second dose of ultraviolet radiation to produce a coated substrate having areas of higher cross-link density and areas of lower cross-link density.

12. The method of claim 9, further comprising pre-exposing to ionizing radiation, at least a portion of the coated substrate subsequently exposed to the first dose of ultraviolet radiation and at least a portion of the coated substrate subsequently shielded from the first dose of ultraviolet radiation to produce a coated substrate having areas of higher cross-link density and areas of lower cross-link density.

13. The method of claim 9, wherein the polysaccharide-based polymer is a poly-glucose-based polymer or a poly-xylose-based polymer.

14. The method of claim 13, wherein the poly-glucose-based polymer is selected from a cellulose-based polymer, a dextran-based polymer, or an amylase-based polymer.

15. The method of claim 9, wherein the polysaccharide-based polymer is selected from the group consisting of hydroxypropylcellulose, methylcellulose, and hydroxyproplymethylcellulose, hydroxyethylcellulose, amylose, dextran, and xylan, or hydrophobically modified derivates thereof

16. A cell culture article produced by the method of claim 9.

17. A cell culture article comprising:
a patterned surface for culturing cells, the surface formed from a coating consisting essentially of a cross-linked polysaccharide-based polymer, wherein the coating is free of cross linking monomers.

18. The article of claim 17, wherein the polysaccharide-based polymer is a poly-glucose-based polymer or a poly-xylose-based polymer.

19. The article of claim 18, wherein the poly-glucose-based polymer is selected from a cellulose-based polymer, a dextran-based polymer, or an amylase-based polymer.

20. The article of claim 17, wherein the polysaccharide-based polymer is selected from the group consisting of hydroxypropylcellulose, methylcellulose, and hydroxyproplymethylcellulose, hydroxyethylcellulose, amylose, dextran, and xylan, or hydrophobically modified derivates thereof
